(19) United States
(12) Patent Application Publication
(10) Pub. No.: US 2012/0136190 A1
(43) Pub. Date: May 31, 2012

(54) PRODUCTION OF 3-METHYLBUT-1-EN BY MEANS OF DEHYDRATION OF 3-METHYLBUTANE-1-OL

(75) Inventors: Alfred Kaizik, Marl (DE); Thomas Quandt, Marl (DE); Michael Grass, Haltern am See (DE); Markus Winterberg, Datteln (DE); Wilfried Bueschken, Haltern am See (DE)

(73) Assignee: EVONIK OXENO GmbH, Marl (DE)

(21) Appl. No.: 13/322,527

(22) PCT Filed: Apr. 28, 2010
(86) PCT No.: PCT/EP2010/055670

§ 371 Date: Jan. 17, 2012

(30) Foreign Application Priority Data
May 29, 2009 (DE) 102009026585.6

(51) Int. Cl. C07C 1/20

(52) U.S. Cl. 585/640

(57) ABSTRACT

The invention relates to a process for preparing 3-methyl-1-butene by dehydration of 3-methyl-1-butanol over an aluminium-containing oxide in the temperature range from 200 to 450° C. in the gas phase or mixed liquid/gas phase, characterized in that an aluminium-containing oxide having a predominantly mesoporous pore structure whose:

a) relative proportion of macropores is less than 15%;

b) distribution of the pore diameter has a monomodal maximum in the range of mesopores from 3.6 to 50 nm;

c) average pore diameter of all pores is in the range of mesopores and macropores from 5 to 20 nm;

d) composition comprises more than 80% of gamma-aluminium oxide, is used.

The present invention relates to the preparation of 3-methyl-1-butene by dehydration of 3-methyl-1-butanol in the presence of a mesoporous aluminium oxide having a uniform pore structure as catalyst.

C₅-Olefins, in particular methylbutenes, are sought-after starting materials in industry. 2-Methyl-1-butene in particular is a starting material which is frequently used in the perfume industry and for preparing isoprene. 3-Methyl-1-butene can be utilized as monomer or comonomer for preparing polymers and copolymers. Although 3-methyl-1-butene is in principle present in C₅ fractions such as light naphtha, the content of 3-methyl-1-butene in such fractions is only about 1-5% by mass. In addition, the isolation of 3-methyl-1-butene from such fractions is relatively complicated.

Some processes for preparing 3-methyl-1-butene are described in the prior art. Methylbutenes can be prepared industrially by means of, for example, metathesis reactions. Thus, DE 199 32 060 describes the preparation of pentenes and methylbutenes from a hydrocarbon stream comprising C₄-olefins.

JP 62-108827 describes the preparation of 3-methyl-1-butene by partial hydrogenation of isoprene.

U.S. Pat. No. 4,234,752 describes the preparation of 3-methyl-1-butene by dehydration of 3-methyl-1-butanol in the presence of a γ-aluminium oxide modified by means of KOH as catalyst. The dehydration is carried out in the gas phase at 330° C. in the presence of nitrogen as carrier gas.

WO 2008/006633 describes the preparation of 3-methyl-1-butene from isobutene-containing olefin mixtures via three process steps. In this process, isobutene is firstly hydroformylated, the hydroformylation product 3-methylbutanal is hydrogenated to 3-methyl-1-butanol and water is subsequently eliminated from the alcohol obtained. The dehydration of 3-methyl-1-butanol to 3-methyl-1-butene is preferably carried out using aluminium oxides modified by means of bases. In the dehydration at 340° C. and 0.15 MPa in the gas phase as described in the example, a γ-aluminium oxide modified by means of 1.5% by mass of barium compounds (calculated as barium oxide) is used as catalyst. The product contains 94.5% by mass of 3-methyl-1-butene as desired product and additionally 3-methyl-2-butene, 2-methyl-1-butene and di(3-methylbutyl) ether as by-products.

According to the prior art as disclosed in U.S. Pat. No. 4,234,752, WO 2005/080302 and WO 2008/006633, the modification by means of bases of the aluminium oxides used in the dehydration of 3-methyl-1-butanol can lead to an improvement in the yields of 3-methyl-1-butene.

It is generally known that unmodified, acidic aluminium oxides can also be used for the dissociation of alcohols to form olefins. The isomer distribution of the internal and terminal olefins isomers obtained by the dehydration of the alcohol is critically dependent on the acidity of the catalyst. Targeted modification of the aluminium oxides by means of bases can improve the selectivity of the formation of terminal a-olefins from primary alcohols.

None of the processes described in the prior art makes it possible to prepare 3-methyl-1-butene from 3-methyl-1-butanol in a simple manner with satisfactory conversions and high selectivities.

It was therefore an object of the present invention to discover a simple and economical process for preparing 3-methyl-1-butene by dehydration of 3-methyl-1-butanol over unmodified aluminium oxides.

Aluminium oxide can occur in various structural forms as a function of the method of preparation and the heat treatment. In general, aluminium oxides are, according to Ullmanns Enzyklopadie der technischen Chemie (VCH Weinheim, volume 7, 1974) divided into three classes, namely the a modification, the y forms and the special forms. Apart from the most thermodynamically stable form of α-aluminium oxide (corundum), the other aluminium oxide modifications are high-surface-area oxides. The γ-aluminium oxide forms are divided further into a γ group and a δ group. The most important representative of the γ group, to which η-aluminium oxide also belongs, is γ-aluminium oxide. The δ group comprises the high-temperature forms such as δ- and ρ-aluminium oxide.

The most important catalytic property of aluminium oxide is based on the presence of acidic sites which are in principle found in every aluminium oxide modification. In addition, the conversion and the selectivity of the chemical reaction is influenced by the pore structure and the internal surface area of the aluminium oxides.

It has now surprisingly been found that 3-methy-1-butene can be prepared in a particularly simple way by dehydration of 3-methyl-1-butanol when a mesoporous aluminium oxide having a uniform pore structure is used as catalyst.

The present invention accordingly provides a process for preparing 3-methyl-1-butene by dehydration of 3-methyl-1-butanol over an aluminium-containing oxide in the temperature range from 200 to 450° C. in the gas phase or mixed liquid/gas phase, characterized in that an aluminium-containing oxide having a predominantly mesoporous pore structure whose:

a) relative proportion of macropores is less than 15%;

b) distribution of the pore diameter has a monomodal maximum in the range of mesopores from 3.6 to 50 nm;

c) average pore diameter of all pores is in the range of mesopores and macropores from 5 to 20 nm;

d) composition comprises more than 80% of gamma-aluminium oxide, is used.

The present invention likewise provides a mixture containing 3-methyl-1-butene and 2-methyl-1-butene and/or 3-methyl-2-butene in which the proportion by mass of 3-methyl-1-butene is at least 90% by mass and the proportion by mass of 2-methyl-1-butene and/or 3-methyl-2-butene is less than 10% by mass.

The process of the invention has the following advantages:

a) inexpensive, commercially available aluminium oxides which are frequently already present in the desired form can be used

b) without after-treatment.

This results in a cost advantage. Furthermore, the catalysts used according to the invention have a high activity and product selectivity.

In the process of the invention for the dehydration of 3-methylbutanol to 3-methyl-1-butene, specific γ-aluminium oxides are used. Preference is given to using mesoporous aluminium oxides having a uniform pore structure. The γ-aluminium oxides used according to the invention have the following features:

the relative proportion of the pore volume made up by macropores (pore diameter from 50 nm to 100 μm) in the pore diameter range from 3.6 nm to 100 μm, determined by Hg porosimetry, is less than 15%. (The relative pore volume of macropores is the ratio of the pore volume over the total macropore range to the total pore volume). In particular, this ratio is less than 10%, very particularly preferably less than 5%.

The average pore diameter of all pores having a diameter of from 3.6 nm to 100 μm is preferably in the range from 5 to 20 nm, very particularly preferably from 6 to 12 nm. (Determined by Hg porosimetry)

The γ-aluminium oxides used according to the invention preferably have a monomodal maximum in the pore diameter range from 3.6 to 50 nm (in particular in the pore diameter range from 5 to 20 nm). (Determined by Hg porosimetry)

The phase composition of the aluminium oxide used according to the invention as determined by X-ray diffraction analysis (XRD) comprises more than 80%, in particular more than 85%, very particularly preferably more than 90%, of γ-aluminium oxide.

The BET surface area of the aluminium oxide used according to the invention is in the range from 120 to 360 m²/g, in particular in the range from 150 to 200 m²/g.

The catalyst used comprises more than 99% by mass of aluminium oxide. It can further comprise titanium dioxide, silicon dioxide and up to 0.2% by mass of alkali metal oxides.

In the characterization of porous materials, including the aluminium oxides according to the invention, pores having pore diameters of less than 2 nm are designated as micropores, pores having pore diameters in the range from 2 to 50 nm are designated as mesopores and pores having diameters of greater than 50 nm are designated as macropores, according to the IUPAC standard (Manual on Catalyst Characterization in Pure & Appl. Chem. Vol. 63, pp.1227, 1991).

To determine the pore radius distribution PRD and the pore volume PV of catalysts in the mesopore and macropore range in accordance with DIN 66133, high-pressure mercury porosimetry is employed. This measurement method is based on the fact that liquid mercury does not wet the pore surface. It penetrates into pores only under an externally applied pressure. This pressure is a function of the pore size. The smallest pore diameter which can be measured is limited by the final mercury pressure employed.

To determine the BET surface area, the pore radius distribution PRD and the pore volume PV in the micropore and mesopore range, nitrogen adsorption at 77 K is employed. Here, the amount of adsorbate (N₂) is determined as a function of the relative pressure by means of volumetric measurements at a constant temperature of 77 K. Adsorption and desorption isotherms are constructed from the data obtained and the BET surface area, the pore radius distribution PRD and the average pore diameter are calculated. To determine the BET surface area in accordance with DIN 66131, the N₂ adsorption isotherms in the relative pressure range (p/po) from 0.1 to 0.3 are employed and the surface area is determined by means of the Brunauer-Emmet-Teller equation. The evaluation is based on the assumption of a monomolecular coverage of the internal surface of the particles, from which the numerical size of the surface area can be calculated.

The determination of the BET surface areas of the aluminium oxides in the present invention was carried out using a sorption apparatus model ASAP 2400 from Micromeritics.

The pore volume PV and the pore radius distribution PRD in the mesopore and macropore range was determined in accordance with DIN 66133 using an Hg porosimeter model Pascal 140/440 from Porotec. The maximum Hg pressure of the measurement station is limited to 400 MPa (4000 bar).

The instrument makes it possible to determine pore volume PV and pore radius distribution PRD of pores having diameters Dp of from 3.6 nm to 100 μm. The relative percentages of mesopore volume having a Dp of from 3.6 to 50 nm and of the macropore volume having Dp>50 nm can be determined from the measured total pore volume.

In the process of the invention, the dehydration can be carried out in the gas phase or the mixed liquid/gas phase. The process can be carried out continuously or batchwise and over suspended catalysts or particulate catalysts arranged in a fixed bed. The elimination of water is preferably carried out in the gas phase or the mixed gas/liquid phase in the temperature range from 200 to 450° C. over solid catalysts because of the ease of separation of the reaction products from the reaction mixture. A continuous dehydration over a catalyst arranged in a fixed bed is particularly preferably carried out.

The catalysts are preferably used in the form of spheres, pellets, cylinders, rod-shaped extrudates or rings.

The dehydration of 3-methyl-1-butanol can, for example, be carried out adiabatically, polytropically or virtually isothermally, i.e. with a temperature difference of typically less than 10° C. The process step can be carried out in one or more stages. In the latter case, all reactors, advantageously tube reactors, can be operated adiabatically or virtually isothermally. It is likewise possible to operate one or more reactors adiabatically and the others virtually isothermally. The elimination of water is preferably carried out in a single pass. However, it can also be carried out with recirculation of product. In the case of operation in a single pass, the specific weight hourly space velocity is from 0.01 to 30 kg of alcohol, preferably from 0.1 to 10 kg of alcohol, per kg of catalyst and per hour. In the elimination of water, the temperature in the catalyst bed is preferably from 200 to 450° C., in particular from 250 to 320° C. The elimination of water (dehydration) can be carried out under reduced pressure, under superatmospheric pressure or at atmospheric pressure.

To achieve a very high selectivity to 3-methyl-1-butene, it has been found to be advantageous for only a partial conversion of the alcohol used to be sought. The conversion in a single pass is preferably limited to from 30 to 90%.

The 3-methyl-1-butene according to the invention, which can, in particular, be obtained by the process of the invention, preferably contains less than or equal to 10% by mass, preferably less than or equal to 1% by mass and particularly preferably from 0.001 to 1% by mass, of 2-methyl-1-butene and/or 3-methyl-2-butene. A preferred mixture contains 3-methyl-1-butene and 2-methyl-1-butene and/or 3-methyl-2-butene, with the proportion by mass of 3-methyl-1-butene being at least 90% by mass and the proportion by mass of 2-methyl-1-butene and/or 3-methyl-2-butene being less than 10% by mass. The mixture preferably comprises at least 99% by mass and particularly preferably from 99.000 to 99.999% by mass of 3-methyl-1-butene and preferably less than or equal to 1% by mass and particularly preferably from 0.001 to 1% by mass of 2-methyl-1-butene and/or 3-methyl-2-butene, with the proportions adding up to 100%.

### EXAMPLES

The following examples illustrate the process of the invention

Commercially available aluminium oxides were used as catalyst for the dehydration of 3-methyl-1-butanol in the examples. The results of the characterization of the catalysts by the above-described measurement methods are shown in Table 1.

TABLE 1
Characterization of the aluminium oxides

| | | Designation of the aluminium oxide | | | |
|---|---|---|---|---|---|
| | | SP 537 | SP 538 F | LD 350 | SA 31132 |
| | | Manufacturer | | | |
| | | Axens | Axens | Alcoa | Saint Gobain |
| spec. PV (total, using cyclohexane) | [cm3/g] | 0.65 | 0.71 | 0.73 | 0.89 |
| spec. PV (PRD Hg, Dp > 3.6 nm) | [cm3/g] | 0.61 | 0.62 | 0.49 | 0.80 |
| spec. PV (PRD Hg, Dp > 3.6 nm-50 nm) | [cm3/g] | 0.59 | 0.60 | 0.25 | 0.37 |
| spec. PV (PRD Hg, Dp > 50 nm/macropores) | [cm3/g] | 0.01 | 0.02 | 0.24 | 0.43 |
| rel. PV of macropores (PRD Hg, Dp > 50 nm) | [%] | 2.14 | 2.43 | 48.67 | 53.81 |
| average Dp (PRD Hg, Dp > 3.6 nm) | [nm] | 8.6 | 9.8 | 39.1 | 63.4 |
| rel. PV of macropores (based on total PV) | [%] | 2.0 | 2.1 | 32.5 | 48.4 |
| rel. PV of mesopores (based on total PV) | [%] | 91.4 | 84.9 | 34.2 | 41.6 |
| rel. PV of micropores (based on total PV) | [%] | 6.6 | 13.0 | 33.3 | 10.0 |
| spec. internal surface area (total/BET) | [m2/g] | 197 | 261 | 355 | 59 |

The aluminium oxides SP 537 and SP 538 F listed in Table 1 are examples of the catalysts used according to the invention. As can be seen from Table 1, they have a high proportion of mesopores. The relative proportion of the total pore volume in the range of pore sizes of from 3.6 nm to 100 μm made up by macropores is less than 5%. In contrast, the two aluminium oxides LD 350 and SA 31132 which are not according to the invention have high proportions of macropores and small proportions of mesopores.

### Example 1

Dehydration of 3-methyl-1-butanol (not according to the invention) 3-Methyl-1-butanol having a purity of 99.81% by mass was reacted over the aluminium oxide catalyst LD 350 in spherical form (2-3 mm spheres) having a bulk density of 0.59 g/cm³ in an electrically heated flow-through fixed-bed reactor. Before entry into the reactor, the liquid starting material was vaporized at 220° C. in an upstream vaporizer. At reaction temperatures in the range from 300 to 330° C., 13.6 g/h of 3-methyl-1-butanol were passed in the gas phase through 23.7 g of catalyst, corresponding to a WHSV of 0.57 h⁻¹. The specific WHSV (weight hourly space velocity) over the catalyst is expressed in gram of starting material per gram of catalyst per hour. The reaction pressure was 0.15 MPa. The gaseous product was cooled in a condenser and collected in a glass receiver. The product had the following composition calculated on a water-free basis:

TABLE 2
Results of the dehydration over the
aluminium oxide catalyst LD 350

| | Reactor Temperature [° C.] | | | |
|---|---|---|---|---|
| | 300 | 310 | 320 | 330 |
| | % by | % by | % by | % by |
| | mass | mass | mass | mass |
| Composition | | | | |
| 3-methyl-1-butene | 14.40 | 22.71 | 33.99 | 41.51 |
| 2-methyl-1-butene | 0.23 | 0.40 | 0.72 | 1.07 |
| 2-methyl-2-butene | 0.66 | 1.18 | 2.12 | 3.13 |
| 3-methyl-2-butanol | 0.04 | 0.04 | 0.04 | 0.03 |
| 3-methyl-1-butanol | 48.08 | 35.52 | 26.89 | 22.32 |
| di(3-methylbutyl) ether | 36.20 | 39.67 | 35.77 | 31.24 |
| miscellaneous/high boilers | 0.39 | 0.47 | 0.48 | 0.69 |
| C5-olefin isomers (normalized) | | | | |
| 3-methyl-1-butene [%] | 94.17 | 93.47 | 92.31 | 90.82 |
| 2-methyl-1-butene [%] | 1.49 | 1.65 | 1.95 | 2.34 |
| 2-methyl-2-butene [%] | 4.34 | 4.88 | 5.75 | 6.84 |

Table 2 shows the composition of the product and also the distribution of the C₅-olefin isomers normalized to 100%. Under the reaction conditions selected, a 3-methyl-1-butene content of about 41.5% by mass was achieved at a reaction temperature of 330° C. As the reaction temperature increases, the formation of the desired product 3-methyl-1-butene increases. The main byproduct formed in the dissociation of 3-methyl-1-butanol is the ether of 3-methyl-1-butanol, viz. di-3-methylbutyl ether (diisoamyl ether).

### Example 2

Dehydration of 3-methyl-1-butanol (not according to the invention) 3-Methyl-1-butanol having a purity of 99.81% by mass was reacted over the aluminium oxide catalyst SA 31132 (extrudates having a diameter of 3 mm and a length of 3-4 mm) having a bulk density of 0.52 g/cm³ in an electrically heated flow-through fixed-bed reactor. Before entry into the reactor, the liquid starting material was, as in Example 1, vaporized at 220° C. in an upstream vaporizer. At reaction temperatures in the range from 300 to 330° C., 13.0 g/h of 3-methyl-1-butanol were passed in the gas phase through 22.0 g of catalyst, corresponding to a WHSV of 0.57 h⁻¹. The reaction pressure was 0.15 MPa. The gaseous product was cooled in a condenser and collected in a glass receiver. The product had the following composition calculated on a water-free basis:

TABLE 3
Results of the dehydration over the
aluminium oxide catalyst SA 31132

| | Reactor Temperature [° C.] | | | |
|---|---|---|---|---|
| | 300 | 310 | 320 | 330 |
| | % by | % by | % by | % by |
| | mass | mass | mass | mass |
| Composition | | | | |
| 3-methyl-1-butene | 8.66 | 17.07 | 28.88 | 41.52 |
| 2-methyl-1-butene | 0.04 | 0.08 | 0.14 | 0.26 |
| 2-methyl-2-butene | 0.09 | 0.23 | 0.45 | 0.89 |
| 3-methyl-2-butanol | 0.03 | 0.00 | 0.03 | 0.05 |
| 3-methyl-1-butanol | 73.94 | 50.52 | 37.36 | 22.52 |
| di(3-methylbutyl) ether | 16.58 | 31.41 | 32.22 | 33.52 |
| miscellaneous/high boilers | 0.65 | 0.69 | 0.92 | 1.26 |
| C5-olefin isomers (normalized) | | | | |
| 3-methyl-1-butene [%] | 98.49 | 98.23 | 98.00 | 97.33 |
| 2-methyl-1-butene [%] | 0.45 | 0.44 | 0.48 | 0.60 |
| 2-methyl-2-butene [%] | 1.07 | 1.33 | 1.51 | 2.07 |

Table 3 shows the composition of the product and also the distribution of the C5-olefin isomers normalized to 100%. As can be seen from Table 3, comparable contents of the desired product 3-methyl-1-butene were achieved over the noninventive catalyst SA 31132 under comparable reaction conditions as in Example 1. The selectivity of the dissociation of 3-methyl-1-butanol is reduced by the formation of di(3-methylbutyl) ether. Under the reaction conditions selected, a 3-methyl-1-butene content of about 41.5% by mass was achieved at a reaction temperature of 330° C.

### Example 3

Dehydration of 3-methyl-1-butanol (according to the invention) 3-Methyl-1-butanol having a purity of 99.81% by mass was reacted over the aluminium oxide catalyst SP 537 in sphere form (1.7-2.1 mm beads) having a bulk density of 0.58 g/cm³ in an electrically heated flow-through fixed-bed reactor. Before entry into the reactor, the liquid starting material was vaporized at 220° C. in an upstream vaporizer. At reaction temperatures in the range from 250 to 300° C., 13.6 g/h of 3-methyl-1-butanol were passed in the gas phase through 26.0 g of catalyst, corresponding to a WHSV of 0.52 h⁻¹. The reaction pressure was, as in Example 1, 0.15 MPa. The gaseous product was cooled in a condenser and collected in a glass receiver. The product had the following composition calculated on a water-free basis:

TABLE 4
Results of the dehydration over the
aluminium oxide catalyst SP 537

| | Reactor Temperature [° C.] | | | | | |
|---|---|---|---|---|---|---|
| | 250 | 260 | 270 | 280 | 290 | 300 |
| | % by | % by | % by | % by | % by | % by |
| Composition | mass | mass | mass | mass | mass | mass |
| 3-methyl-1-butene | 38.79 | 56.65 | 87.45 | 86.08 | 72.87 | 54.25 |
| 2-methyl-1-butene | 0.09 | 0.19 | 0.76 | 2.60 | 5.87 | 10.23 |
| 2-methyl-2-butene | 0.66 | 1.43 | 4.27 | 10.82 | 20.88 | 34.99 |
| 3-methyl-2-butanol | 0.02 | 0.02 | 0.01 | 0.00 | 0.00 | 0.00 |
| 3-methyl-1-butanol | 24.60 | 14.95 | 3.91 | 0.02 | 0.00 | 0.00 |
| di(3-methylbutyl) ether | 35.47 | 26.36 | 3.28 | 0.00 | 0.00 | 0.00 |
| miscellaneous/high boilers | 0.37 | 0.39 | 0.32 | 0.47 | 0.38 | 0.52 |
| C5-olefin isomers | | | | | | |
| (normalized) | | | | | | |
| 3-methyl-1-butene [%] | 98.10 | 97.20 | 94.56 | 86.51 | 73.15 | 54.54 |
| 2-methyl-1-butene [%] | 0.22 | 0.33 | 0.82 | 2.62 | 5.89 | 10.29 |
| 2-methyl-2-butene [%] | 1.68 | 2.46 | 4.62 | 10.87 | 20.96 | 35.18 |

As can be seen from Table 4, contents of 3-methyl-1-butene of above 56% by mass were achieved over the catalyst according to the invention even at low reaction temperatures above 260° C. The optimal temperature range for achieving high yields of 3-methyl-1-butene at the chosen WHSV over the catalyst is 270-280° C. Very high 3-methyl-1-butene contents of above 86% by mass are achieved in this temperature range. At a reaction temperature of 280° C. and above, the starting material 3-methyl-1-butanol is converted completely into C₅-olefins and water over the catalyst according to the invention. The proportion of 3-methyl-1-butene in the C₅-olefin mixture decreases with increasing temperature at a constant WHSV over the catalyst, as expected due to the isomerization of the 3-methyl-1-butene formed to internal C₅-olefin isomers.

Comparison of the results in Table 4 with the results in Tables 2 and 3 shows that the catalyst SP 537 according to the invention displays a significantly higher activity and selectivity compared to the noninventive catalysts.

### Example 4

Dehydration of 3-methyl-1-butanol (according to the invention) 3-Methyl-1-butanol having a purity of 99.81% by mass was reacted over the aluminium oxide catalyst SP 538 F in trilobe form (1.8 mm trilobes) having a bulk density of 0.55 g/cm³ in an electrically heated flow-through fixed-bed reactor. The pore structure of the SP catalyst 587 F is largely comparable with the pore structure of the catalyst SP 537 used in Example 3 (see Table 1). Before entry into the reactor, the liquid starting material was vaporized at 220° C. in an upstream vaporizer. At reaction temperatures in the range from 280 to 300° C., 13.6 g/h of 3-methyl-1-butanol were passed in the gas phase through 24.7 g of catalyst, corresponding to a WHSV of 0.55 h⁻¹. The reaction pressure was, as in preceding examples, 0.15 MPa. The gaseous product was cooled in a condenser and collected in a glass receiver. The product had the following composition calculated on a water-free basis:

TABLE 5
Results of the dehydration over the
aluminium oxide catalyst SP 538 F

Reactor Temperature [° C.]

| | 280 | 290 | 300 |
|---|---|---|---|
| | % by | % by | % by |
| | mass | mass | mass |
| Composition | | | |
| 3-methyl-1-butene | 87.44 | 91.37 | 84.51 |
| 2-methyl-1-butene | 0.54 | 1.71 | 3.40 |
| 2-methyl-2-butene | 3.04 | 6.58 | 11.77 |
| 3-methyl-2-butanol | 0.02 | 0.00 | 0.00 |
| 3-methyl-1-butanol | 5.07 | 0.06 | 0.00 |
| di(3-methylbutyl) ether | 3.56 | 0.01 | 0.00 |
| miscellaneous/high boilers | 0.34 | 0.27 | 0.33 |
| C5-olefin isomers (normalized) | | | |
| 3-methyl-1-butene [%] | 96.06 | 91.69 | 84.78 |
| 2-methyl-1-butene [%] | 0.60 | 1.71 | 3.41 |
| 2-methyl-2-butene [%] | 3.34 | 6.60 | 11.81 |

The catalytic behaviour of the catalyst SP 538 F, e.g. activity and selectivity, resembles the behaviour of the catalyst SP 537. In the temperature range from 280 to 300° C., very high contents of 3-methyl-1-butene of above 84% by mass were achieved at a comparable WHSV. Under the conditions selected, the yield maximum is at a reaction temperature of 290° C. with a 3-methyl-1-butene content of about 91.4% by mass. At this temperature, the 3-methyl-1-butanol is converted completely into C₅-olefins and water over the catalyst SP 538 F according to the invention. In contrast to the noninventive macroporous catalysts in Examples 1 and 2, no di(3-methylbutyl) ether is formed at temperatures above 290° C.

1. Process for preparing 3-methyl-1-butene by dehydration of 3-methyl-1-butanol over an aluminium-containing oxide in the temperature range from 200 to 450° C. in the gas phase or mixed liquid/gas phase, characterized in that an aluminium-containing oxide having a predominantly mesoporous pore structure whose:
a) relative proportion of macropores is less than 15%;
b) distribution of the pore diameter has a monomodal maximum in the range of mesopores from 3.6 to 50 nm;
c) average pore diameter of all pores is in the range of mesopores and macropores from 5 to 20 nm;
d) composition comprises more than 80% of gamma-aluminium oxide, is used.

2. Process according to claim 1, characterized in that
the relative proportion of macropores is less than 5%.

3. Process according to claim 2, characterized in that
the average pore diameter of all pores in the range of mesopores and macropores is from 6 to 12 nm.

4. Process according to claim 3, characterized in that
the aluminium-containing oxide comprises more than 90% of gamma-aluminium oxide.
